(19) United States
(12) Patent Application Publication
(10) Pub. No.: US 2026/0314991 A1
(43) Pub. Date: Oct. 15, 2026

(54) VIRTUAL REALITY WALKING BALANCE ASSESSMENT SYSTEM USING FIRM AND FOAM SURFACES FOR SENSORY CONTRIBUTIONS TO WALKING BALANCE

(71) Applicant: Kwadwo Appiah-Kubi, Potsdam, NY (US)

(72) Inventor: Kwadwo Appiah-Kubi, Potsdam, NY (US)

(73) Assignee: Clarkson University, Potsdam, NY (US)

(21) Appl. No.: 19/647,227

(22) Filed: Apr. 14, 2026

Related U.S. Application Data
(60) Provisional application No. 63/788,602, filed on Apr. 14, 2025.

(51) Int. Cl. A61B 5/00; A61B 5/11; G06F 3/01

(52) U.S. Cl. CPC A61B 5/4023; A61B 5/1114; A61B 5/112; A61B 5/6803; G06F 3/012

(57) ABSTRACT

A virtual reality walking balance assessment system includes a head-mounted display configured to present an interactive virtual environment to a participant during over-ground walking and to generate head-tracking data representing motion of the head-mounted display during the over-ground walking. A walking test protocol specifies a plurality of visual scene conditions and specifies a first walking surface condition and a second walking surface condition that is more compliant than the first walking surface condition. One or more processors obtain the head-tracking data and compute one or more gait balance metrics and one or more sensory contribution ratios indicative of relative contributions of visual input, vestibular input, and somatosensory input to walking balance. In some embodiments, the plurality of visual scene conditions includes a stable scene, a dark scene, and a rotating scene, and the first walking surface condition is a firm surface and the second walking surface condition is a foam surface. In some embodiments, the virtual reality walking balance assessment system can additionally be configured as a rehabilitation system for improving walking balance after completion of an assessment protocol.

### CROSS-REFERENCE TO RELATED APPLICATION

The present application relates and claims priority to U.S. Provisional Application No. 63/788,602, filed Apr. 14, 2025, the entirety of which is hereby incorporated by reference.

### BACKGROUND

The present disclosure relates to virtual reality (VR) walking balance assessment systems and, more particularly, to a virtual reality (VR) walking balance assessment system using firm and foam surfaces for assessing sensory contributions to walking balance.

Walking balance is a dynamic function that depends on the coordinated contribution of multiple sensory systems, including visual, somatosensory, and vestibular inputs. Information from these sensory systems is integrated by the nervous system to support orientation, stability, and motor control during locomotion. When one or more of these sensory inputs is reduced, distorted, or improperly integrated, an individual may experience walking dysfunction, dizziness, increased fall risk, and decreased mobility.

Assessment of balance is used in rehabilitation and clinical practice to help identify functional deficits and to guide patient management. Existing balance evaluations, however, are often directed to static or standing postural control rather than balance during walking. Because locomotion involves continuous movement and changing environmental demands, the sensory requirements associated with walking balance can differ from those involved in quiet standing.

Some gait-related balance assessments have been developed in research settings to examine the effect of altered sensory conditions during locomotion. However, such approaches may rely on large or complex equipment, may be costly, and may be difficult to implement in routine clinical environments. As a result, clinicians may have limited access to practical tools for quantitatively evaluating how different sensory systems contribute to walking balance.

Accordingly, there is a need in the art for systems and methods that can assess walking balance in a clinically accessible manner while providing quantitative information regarding sensory contributions during gait.

### SUMMARY OF THE INVENTION

The present disclosure is directed to a virtual reality walking balance assessment system, a method of assessing sensory contributions to walking balance, and a non-transitory computer-readable medium for assessing sensory contributions to walking balance during over-ground walking.

According to an aspect, a virtual reality walking balance assessment system comprises a head-mounted display configured to present an interactive virtual environment to a participant during over-ground walking and to generate head-tracking data representing motion of the head-mounted display during the over-ground walking, a walking surface arrangement providing at least a first walking surface condition and a second walking surface condition that is more compliant than the first walking surface condition, and one or more processors configured to cause administration of a walking test protocol in which the interactive virtual environment is presented under a plurality of visual scene conditions while the participant walks under the first walking surface condition and the second walking surface condition, obtain the head-tracking data for the walking test protocol, and compute, from the head-tracking data, one or more gait balance metrics and one or more sensory contribution ratios indicative of relative contributions of visual input, vestibular input, and somatosensory input to walking balance. According to an embodiment, the interactive virtual environment can comprise a virtual hallway. According to an embodiment, the plurality of visual scene conditions can comprise a stable scene, a dark scene, and a rotating scene. According to an embodiment, the first walking surface condition can be a firm surface and the second walking surface condition can be a foam surface. According to an embodiment, the one or more gait balance metrics can comprise sway path velocity. According to an embodiment, the one or more processors may be configured to compute positional displacement as a gait balance metric. According to an embodiment, the head-tracking data can represent head kinematics comprising at least one of position, velocity, or acceleration of the head-mounted display. According to an embodiment, the one or more processors may be configured to compute a stepwise deviation angle based on a ratio of a medio-lateral head position component to an anterior-posterior head position component. According to an embodiment, the one or more sensory contribution ratios can comprise at least one of a somatosensory ratio, a visual ratio, a vestibular ratio, or a preference ratio. According to an embodiment, the system can further comprise a protocol user interface configured to receive one or more protocol settings comprising at least one of a test type, a test length, a warning boundary, a lower angle limit, an upper angle limit, a rotation speed multiplier, or a recording name prefix.

According to another aspect, a method of assessing sensory contributions to walking balance comprises presenting, by a head-mounted display, an interactive virtual environment to a participant during over-ground walking, selecting a walking test protocol that specifies a plurality of visual scene conditions and specifies a first walking surface condition and a second walking surface condition that is more compliant than the first walking surface condition, obtaining head-tracking data representing motion of the head-mounted display while the participant walks under the first walking surface condition and the second walking surface condition, and computing, from the head-tracking data, one or more gait balance metrics and one or more sensory contribution ratios indicative of relative contributions of visual input, vestibular input, and somatosensory input to walking balance. According to an embodiment, selecting the walking test protocol may comprise selecting the first walking surface condition as a firm surface and selecting the second walking surface condition as a foam surface. According to an embodiment, selecting the plurality of visual scene conditions may comprise selecting a stable scene, a dark scene, and a rotating scene. According to an embodiment, computing the one or more gait balance metrics may comprise computing sway path velocity. According to an embodiment, computing the one or more gait balance metrics may comprise computing a stepwise deviation angle. According to an embodiment, the method can further comprise defining a virtual walking boundary and providing an alert in response to detecting deviation beyond the virtual walking boundary.

According to a further aspect, a non-transitory computer-readable medium stores instructions that, when executed by one or more processors of a virtual reality walking balance assessment system, cause the virtual reality walking balance assessment system to present, by a head-mounted display, an interactive virtual environment to a participant during over-ground walking under a walking test protocol that specifies a plurality of visual scene conditions and specifies a first walking surface condition and a second walking surface condition that is more compliant than the first walking surface condition, obtain head-tracking data representing motion of the head-mounted display while the participant walks under the first walking surface condition and the second walking surface condition, and compute, from the head-tracking data, one or more gait balance metrics and one or more sensory contribution ratios indicative of relative contributions of visual input, vestibular input, and somatosensory input to walking balance. According to an embodiment, the instructions can further cause the virtual reality walking balance assessment system to display the one or more gait balance metrics on a display. According to an embodiment, the instructions can further cause the virtual reality walking balance assessment system to store or export the one or more gait balance metrics to a secure cloud-based database. According to an embodiment, the instructions can further cause the virtual reality walking balance assessment system to generate a recommendation for a rehabilitation strategy based on the one or more sensory contribution ratios.

These and other aspects will be apparent from the embodiments described below.

### BRIEF DESCRIPTION OF THE DRAWINGS

The present disclosure will be more fully understood and appreciated by reading the following Detailed Description in conjunction with the accompanying drawings, in which:

FIG. 1 shows a GaitSIT VR hallway interface for the virtual reality walking balance assessment system, in accordance with an embodiment.

FIG. 2 shows six GaitSIT assessment conditions using stable VR scenes, dark VR scenes, and rotating VR scenes on firm and foam surfaces, in accordance with an embodiment.

FIG. 3 shows a GaitSIT menu page for configuring a customizable test protocol for assessing sensory contributions to walking balance, in accordance with an embodiment.

### DETAILED DESCRIPTION

The present disclosure describes a virtual reality (VR) walking balance assessment system and related methods, including GaitSIT (Gait Sensory Interaction Test), by way of illustrative and non-limiting examples. The reader will understand that the embodiments described herein are provided to explain the principles of the present disclosure and are not intended to limit the scope of the appended claims. Accordingly, particular arrangements, configurations, parameters, materials, dimensions, sequences, and described components can vary, and various rearrangements, substitutions, modifications, and equivalents can be used while still falling within the scope of the present disclosure. In some embodiments, some features can be optional and are not required to be present in various embodiments. In some embodiments, features described in connection with one embodiment can be combined with features described in connection with another embodiment unless such features are incompatible. In addition, certain structures, operations, and implementation details that are well-understood in the field may be omitted or described at a high level so as not to obscure the present disclosure.

The present disclosure relates to virtual reality walking balance assessment systems and, more particularly, to a VR walking balance assessment system configured for assessing sensory contributions to walking balance. In some embodiments, the system can include a VR application configured to generate an interactive virtual environment, a VR headset configured to collect real-time head position and motion data using built-in inertial sensors, and a set of conditions that systematically manipulate visual, vestibular, and somatosensory inputs during walking balance assessment, including a stable VR scene, a dark VR scene, a rotating VR scene, and conditions performed on firm and foam surfaces to modify somatosensory input. In some embodiments, the present disclosure can further relate to quantitative assessment of walking balance using head-tracking motion data, sway path velocity, positional displacement, and sensory contribution ratios, and can provide clinically accessible tools for different populations and clinical applications.

Walking balance depends on coordinated visual, somatosensory, and vestibular input during locomotion, yet balance assessment tools may not adequately evaluate how those sensory systems contribute while a person is walking. Assessments directed primarily to static or standing postural control can fail to capture the changing sensory demands of gait, and walking-based approaches can be difficult to implement in routine clinical environments when they rely on large, complex, or costly equipment. As a result, a clinician may lack a clinically accessible and quantitative way to determine whether altered walking balance is associated more strongly with visual, somatosensory, or vestibular dysfunction.

To address these issues, the present disclosure provides a virtual reality walking balance assessment system that can systematically challenge/perturb sensory conditions during gait while collecting motion information from a head-mounted device. In some embodiments, a virtual reality application presents different visual scenes, such as stable, dark, or rotating scenes, and the assessment can be performed on firm and foam surfaces so that visual, vestibular, and somatosensory inputs are challenged in different combinations during walking. Head-tracking data, including position and rotational information, can be processed to derive quantitative walking balance measures, such as sway path velocity, positional displacement, and sensory contribution ratios, thereby enabling identification of relative sensory contributions to gait stability. In some embodiments, the system can further provide boundary feedback, real-time visualization, and customizable protocol settings so that the assessment can be adapted for different users and clinical applications while remaining portable and practical to administer.

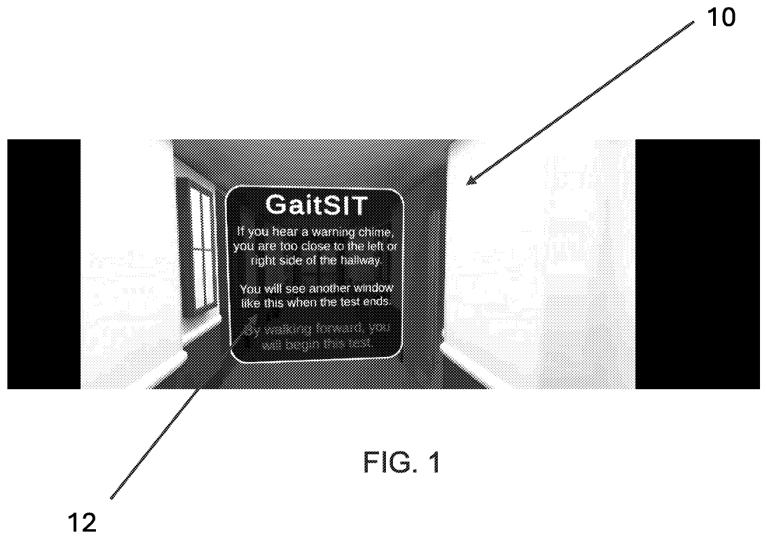

FIG. 1 shows a GaitSIT VR hallway 10 and visual and audio instructions 12 for a virtual reality (VR) walking balance assessment system. In the illustrated embodiment, the GaitSIT VR hallway 10 is presented by a VR application as an interactive virtual environment through which a subject walks during assessment of sensory contributions to walking balance. The visual and audio instructions 12 are presented within the GaitSIT VR hallway 10 to guide the subject at initiation of a test or condition.

The GaitSIT VR hallway 10 may define a virtual hallway or virtual hallway/walkway extending in a forward direction to provide a standardized visual scene during walking. In some embodiments, the GaitSIT VR hallway 10 may correspond to a stable VR scene and may cooperate with a VR headset that collects head-tracking data, including position and rotational information, while the subject moves through the interactive virtual environment.

The visual and audio instructions 12 may provide condition-specific guidance before or during walking and may notify the subject when a condition is about to begin and when it ends. In some embodiments, the visual and audio instructions 12 may include visual prompts, voice-over instructions, audio cues, or combinations thereof. Where a virtual boundary or boundary alert is used, the visual and audio instructions 12 may further cooperate with audio and/or visual feedback to indicate that the subject has deviated from a predefined walking boundary.

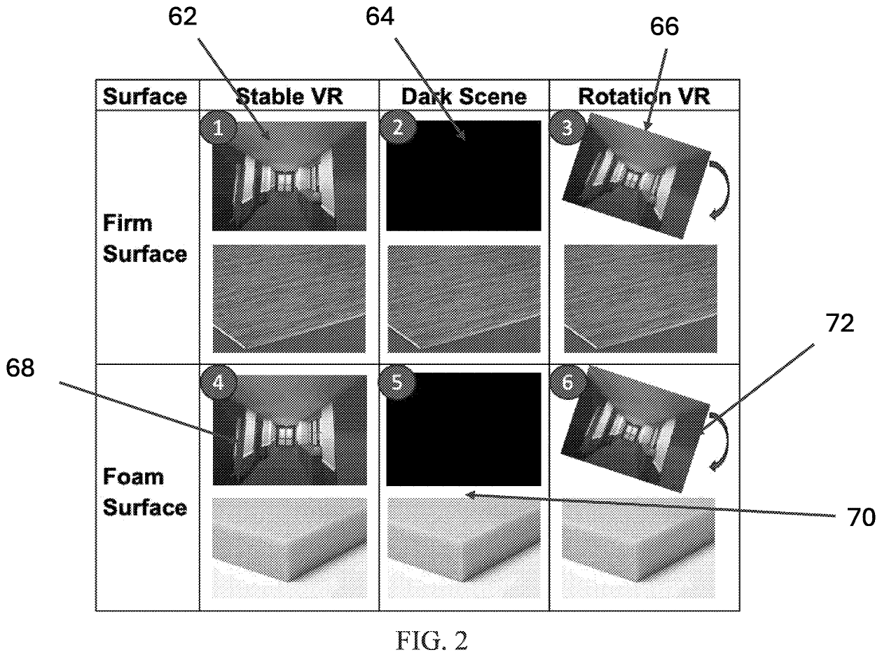

FIG. 2 shows six GaitSIT assessment conditions including a Stable VR on Firm Surface 62, a Dark Scene on Firm Surface 64, a Rotation VR on Firm Surface 66, a Stable VR on Foam Surface 68, a Dark Scene on Foam Surface 70, and a Rotation VR on Foam Surface 72. In the illustrated embodiment, the six GaitSIT assessment conditions are arranged to combine different visual scenes with firm surface and foam surface walking conditions so that visual, vestibular, and somatosensory inputs are challenged in different combinations during walking balance assessment.

The Stable VR on Firm Surface 62, the Dark Scene on Firm Surface 64, and the Rotation VR on Firm Surface 66 are conditions performed while the subject walks on a firm surface. The Stable VR on Firm Surface 62 may present a stable VR scene during walking. The Dark Scene on Firm Surface 64 may reduce or remove visual input during walking on the firm surface. The Rotation VR on Firm Surface 66 may present a rotating VR scene while the subject remains on the firm surface, thereby increasing visual and vestibular challenge relative to a stable VR scene.

The Stable VR on Foam Surface 68, the Dark Scene on Foam Surface 70, and the Rotation VR on Foam Surface 72 are conditions performed while the subject walks on a foam surface. The foam surface may alter somatosensory input relative to the firm surface. The Stable VR on Foam Surface 68 may present a stable VR scene while somatosensory input is modified by the foam surface. The Dark Scene on Foam Surface 70 may combine the foam surface with reduced visual input. The Rotation VR on Foam Surface 72 may combine the foam surface with a rotating VR scene so that the subject is assessed under concurrent visual, vestibular, and somatosensory challenge.

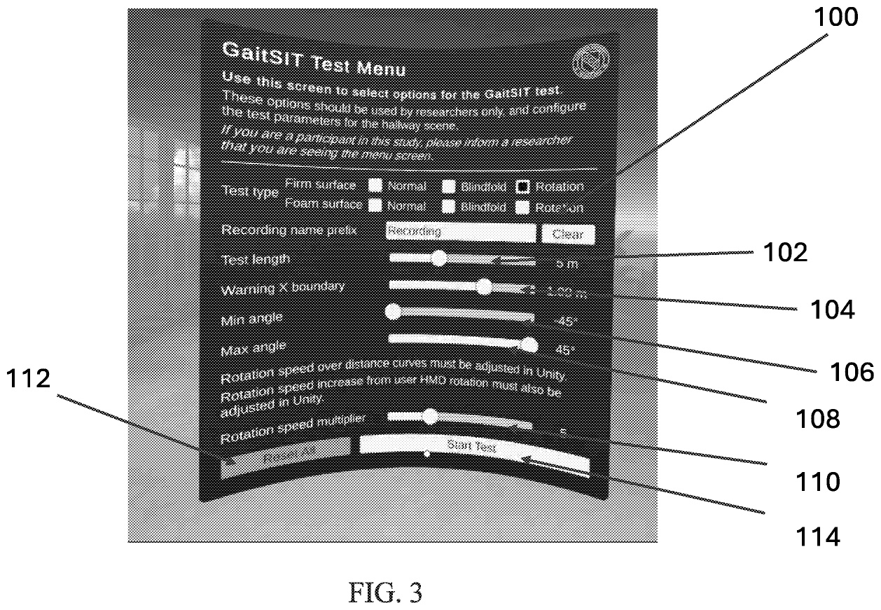

FIG. 3 shows a GaitSIT menu page including a Test type 100, a Recording name prefix 102, a Test length 104, a Warning X boundary 106, a Min angle 108, a Max angle 110, a Rotation speed multiplier 112, a Reset All 114, and a Start Test 116. In the illustrated embodiment, the GaitSIT menu page is generated by the VR application to configure a customizable test protocol for assessing sensory contributions to walking balance.

The Test type 100 may allow selection of one or more GaitSIT conditions for a given assessment protocol. The Recording name prefix 102 may receive a participant name or ID, or another recording identifier, for association with collected assessment data. The Test length 104 may define a walking distance or test duration for the assessment. The Warning X boundary 106 may define a virtual boundary used for boundary alert during walking.

The Min angle 108 and the Max angle 110 may define a range of angular displacement for the rotating VR scene. The Rotation speed multiplier 112 may define or adjust a rotation speed for the rotating VR scene. The Reset All 114 may restore default or previously set parameters, and the Start Test 116 may initiate execution of the customizable test protocol using the selected GaitSIT condition and parameter settings.

The head-mounted display presents an interactive virtual environment during over-ground walking and generates head-tracking data representing motion of the head-mounted display. This feature allows visual conditions to be controlled during actual walking rather than during a standing-only task, and also enables objective capture of head motion so that walking balance can be assessed from measured kinematic behavior under the presented scene.

The walking surface arrangement provides a first walking surface condition and a second walking surface condition that is more compliant than the first. The more compliant surface allows the assessment to alter the reliability of somatosensory input from the feet relative to a firm surface, which enables balance behavior to be observed under different levels of surface support during over-ground walking.

The one or more processors administer the walking test protocol under a plurality of visual scene conditions while the participant walks under both walking surface conditions. This arrangement allows visual and surface-related sensory challenges to be combined in a controlled set of test conditions, which enables condition-specific comparisons of walking balance responses across different sensory demands.

The one or more processors compute, from the head-tracking data, one or more gait balance metrics and one or more sensory contribution ratios indicative of relative contributions of visual input, vestibular input, and somatosensory input to walking balance. This computation enables quantitative characterization of walking balance and allows the relative influence of different sensory inputs on gait stability to be distinguished from the measured head motion.

Presenting the interactive virtual environment to the participant during over-ground walking allows the participant to be exposed to controlled visual scenes while performing a walking task, and obtaining head-tracking data representing motion of the head-mounted display enables the method to use objective motion information acquired during the walking trial rather than relying only on subjective observation.

Selecting the walking test protocol to specify a plurality of visual scene conditions together with the first walking surface condition and the more compliant second walking surface condition allows the method to impose different combinations of visual and somatosensory challenge. This step enables the assessment to probe how walking balance changes when sensory conditions are systematically varied across the protocol.

Obtaining head-tracking data while the participant walks under the first walking surface condition and the second walking surface condition allows the method to capture condition-dependent differences in head-motion behavior across firm and more compliant support conditions. This step enables direct comparison of balance-related motion responses under different surface-related sensory contexts.

Computing the gait balance metrics and the sensory contribution ratios from the head-tracking data allows the method to produce quantitative outputs associated with walking balance performance and relative sensory reliance. This step enables identification of how visual, vestibular, and somatosensory inputs contribute to gait balance within the tested protocol.

The stored instructions cause the virtual reality walking balance assessment system to present the interactive virtual environment during over-ground walking under a walking test protocol having multiple visual scene conditions and first and second walking surface conditions of different compliance. This arrangement allows the system to carry out a standardized walking assessment in which sensory challenges are introduced in a controlled manner during over-ground locomotion.

The stored instructions cause the system to obtain head-tracking data representing motion of the head-mounted display while the participant walks under the first walking surface condition and the more compliant second walking surface condition. This feature allows the system to capture objective head-motion measurements across the different test conditions so that balance-related changes associated with the protocol can be evaluated from recorded data.

The stored instructions cause the system to compute one or more gait balance metrics and one or more sensory contribution ratios from the head-tracking data. This computation enables automated generation of quantitative indicators of walking balance and of the relative contributions of visual, vestibular, and somatosensory input, thereby supporting repeatable assessment outputs across the defined test conditions.

In some embodiments, the virtual reality walking balance assessment system can additionally be configured as a rehabilitation system for improving walking balance after completion of an assessment protocol or during repeated use over multiple sessions. The one or more processors can receive movement data from the participant during over-ground walking, including head-tracking data obtained from the head-mounted display and, in some embodiments, movement data from one or more additional sensors. The movement data can be processed to determine one or more gait balance metrics and one or more sensory contribution ratios indicative of relative reliance on visual input, vestibular input, and somatosensory input during walking. Based on the gait balance metrics and the sensory contribution ratios, the one or more processors can identify a sensory deficit profile for the participant and can select, generate, or modify one or more rehabilitation tasks presented within the interactive virtual environment.

The sensory deficit profile can characterize walking balance impairment associated with excessive visual dependence, excessive vestibular dependence, excessive somatosensory dependence, impaired sensory reweighting, or other balance-related dysfunction reflected in the measured gait balance metrics and sensory contribution ratios. In some embodiments, the one or more processors can compare the participant's performance across different visual scene conditions and across different walking surface conditions to determine how walking stability changes when visual input is reduced, when visual input is altered by scene motion, or when somatosensory input is altered by a compliant walking surface. The identified sensory deficit profile can be used to determine a participant-specific rehabilitation program that preferentially targets the sensory system or sensory integration deficit associated with the measured walking instability.

In some embodiments, the rehabilitation tasks can comprise walking under altered visual scene conditions within the interactive virtual environment. For example, the rehabilitation tasks can comprise walking under a stable scene, a dark scene, a rotating scene, a reduced-visibility scene, a conflicting visual scene, a moving visual scene, or an optokinetic visual stimulation scene. In some embodiments, the rehabilitation tasks can comprise walking on a compliant surface, including a foam surface, to modify somatosensory input during locomotion. In some embodiments, the rehabilitation tasks can combine altered visual scene conditions and altered walking surface conditions so that the participant practices walking while adapting to controlled changes in visual and somatosensory information.

In some embodiments, the rehabilitation tasks can further comprise simultaneous head movements during walking. The head movements can include horizontal head turns, vertical head turns, diagonal head turns, or combinations thereof. Such head movement tasks can be used to increase vestibular challenge during locomotion and to train sensory integration while the participant continues walking through the interactive virtual environment. In some embodiments, the rehabilitation tasks can additionally or alternatively include turning, stepping, reaching, weight shifting, directional changes, obstacle negotiation, or combinations thereof during walking. In some embodiments, the rehabilitation tasks can further include dual-task walking activities in which the participant performs a cognitive task simultaneously with walking.

The one or more processors can be configured to progressively modify the rehabilitation tasks according to participant performance during a session and across multiple sessions. Progressive modification can include increasing walking speed, increasing walking duration, increasing visual complexity, increasing head movement frequency, reducing external support, increasing surface compliance, or otherwise increasing task difficulty when the participant demonstrates improved walking stability. In some embodiments, the one or more processors can determine changes in gait balance metrics over multiple rehabilitation sessions and can use the determined changes to modify the rehabilitation tasks over time. Accordingly, the rehabilitation program can be adaptive and participant-specific rather than fixed.

In some embodiments, the one or more processors can provide real-time feedback regarding walking performance during execution of the rehabilitation tasks. The real-time feedback can comprise visual feedback presented by the head-mounted display, auditory feedback, haptic feedback, or combinations thereof. For example, the system can provide an alert when the participant deviates beyond a defined walking boundary, can provide cues to maintain alignment within the virtual hallway, or can provide prompts associated with head movement timing, stepping performance, or task completion. In some embodiments, the system can record session-specific performance information and can present summary results that indicate whether the participant's walking balance metrics or sensory contribution ratios are improving over time.

The rehabilitation-related embodiments described herein can be used with a variety of participant populations, including older adults and individuals having vestibular dysfunction, stroke, mild traumatic brain injury, multiple sclerosis, Parkinson disease, joint replacement, osteoarthritis, musculoskeletal injury, or other orthopedic, neurological, or balance-related conditions affecting walking stability. In some embodiments, the system can be configured for use in a clinic, a rehabilitation facility, a home environment, or a community setting. Because the disclosed system uses a head-mounted display and programmable test and rehabilitation conditions, the system can provide a portable platform for both assessment of sensory contributions to walking balance and individualized rehabilitation directed to identified walking balance deficits. In some embodiments, a method of rehabilitating walking balance can comprise presenting the interactive virtual environment to a participant during over-ground walking, obtaining movement data from the participant during walking, computing one or more gait balance metrics and one or more sensory contribution ratios, identifying a sensory deficit profile based on the one or more gait balance metrics and sensory contribution ratios, and modifying one or more rehabilitation tasks in response to the sensory deficit profile. Modifying the one or more rehabilitation tasks can comprise changing a visual scene condition, a walking surface condition, a head movement task, a walking speed, a walking duration, or a level of task difficulty. In some embodiments, a non-transitory computer-readable medium can store instructions that, when executed by one or more processors, cause the one or more processors to receive movement data from a participant during over-ground walking, compute one or more gait balance metrics and one or more sensory contribution ratios, identify a sensory deficit profile, and generate or modify a rehabilitation program based on the sensory deficit profile.

The disclosed virtual reality rehabilitation system combines immersive visual presentation, controlled walking surface conditions, and movement-based analysis during over-ground walking. This arrangement allows walking balance to be assessed and trained in a task context that reflects actual ambulation while still applying controlled sensory challenges. By relating measured walking behavior to visual, vestibular, and somatosensory demands, the system enables rehabilitation tasks to be directed to participant-specific balance deficits rather than applying a fixed training regimen.

The head-mounted display presents an interactive virtual environment during over-ground walking and generates head-tracking data representing participant motion during the walking task. This feature allows standardized visual conditions to be introduced while objective motion information is captured from the same walking activity, so gait behavior can be quantified under stable, reduced-visibility, or visually perturbed scene conditions. The use of head-tracking data during actual walking also enables condition-specific changes in balance behavior to be measured without relying on observational assessment alone.

The walking surface arrangement provides multiple walking surface conditions having different compliance characteristics, such as a firmer surface condition and a more compliant surface condition. This feature allows somatosensory input from the feet and lower limbs to be challenged in a controlled manner during walking, so the participant's balance response can be evaluated or trained under different support conditions. Providing surfaces with different compliance also enables graded progression of task difficulty and supports combined visual and surface-based sensory challenges within the same rehabilitation program.

The processor configuration obtains movement data during over-ground walking, determines gait balance metrics and sensory contribution ratios, identifies a sensory deficit profile from those outputs, and selects or modifies rehabilitation tasks in the interactive virtual environment based on that profile. This processing allows the system to convert measured walking behavior into quantitative indications of relative reliance on visual, vestibular, and somatosensory input, which supports selection of rehabilitation tasks that correspond to the participant's observed balance limitations. The ability to modify the rehabilitation tasks based on the identified profile and subsequent performance further enables progressive, participant-specific treatment as walking balance changes over time.

What is claimed is:

1. A virtual reality walking balance assessment system comprising:
a head-mounted display configured to present an interactive virtual environment to a participant during over-ground walking and to generate head-tracking data representing motion of the head-mounted display during the over-ground walking;
a walking surface arrangement providing at least a first walking surface condition and a second walking surface condition that is more compliant than the first walking surface condition; and
one or more processors configured to cause administration of a walking test protocol in which the interactive virtual environment is presented under a plurality of visual scene conditions while the participant walks under the first walking surface condition and the second walking surface condition, obtain the head-tracking data for the walking test protocol, and compute, from the head-tracking data, one or more gait balance metrics and one or more sensory contribution ratios indicative of relative contributions of visual input, vestibular input, and somatosensory input to walking balance.

2. The system of claim 1, wherein the interactive virtual environment comprises a virtual hallway.

3. The system of claim 1, wherein the plurality of visual scene conditions comprises a stable scene, a dark scene, and a rotating scene.

4. The system of claim 1, wherein the first walking surface condition is a firm surface and the second walking surface condition is a foam surface.

5. The system of claim 1, wherein the one or more gait balance metrics comprise sway path velocity.

6. The system of claim 1, wherein the one or more processors are configured to compute positional displacement as a gait balance metric.

7. The system of claim 1, wherein the head-tracking data represents head kinematics comprising at least one of position, velocity, or acceleration of the head-mounted display.

8. The system of claim 1, wherein the one or more processors are configured to compute a stepwise deviation angle based on a ratio of a medio-lateral head position component to an anterior-posterior head position component.

9. The system of claim 1, wherein the one or more sensory contribution ratios comprise at least one of a somatosensory ratio, a visual ratio, a vestibular ratio, or a preference ratio.

10. The system of claim 1, further comprising a protocol user interface configured to receive one or more protocol settings comprising at least one of a test type, a test length, a warning boundary, a minimum angle, a maximum angle, a rotation speed multiplier, or a recording name prefix.

11. A method of assessing sensory contributions to walking balance, the method comprising presenting, by a head-mounted display, an interactive virtual environment to a participant during over-ground walking, selecting a walking test protocol that specifies a plurality of visual scene conditions and specifies a first walking surface condition and a second walking surface condition that is more compliant than the first walking surface condition, obtaining head-tracking data representing motion of the head-mounted display while the participant walks under the first walking surface condition and the second walking surface condition, and computing, from the head-tracking data, one or more gait balance metrics and one or more sensory contribution ratios indicative of relative contributions of visual input, vestibular input, and somatosensory input to walking balance.

12. The method of claim 11, wherein selecting the walking test protocol comprises selecting the first walking surface condition as a firm surface and selecting the second walking surface condition as a foam surface.

13. The method of claim 11, wherein selecting the plurality of visual scene conditions comprises selecting a stable scene, a dark scene, and a rotating scene.

14. The method of claim 11, wherein computing the one or more gait balance metrics comprises computing sway path velocity.

15. The method of claim 11, wherein computing the one or more gait balance metrics comprises computing a stepwise deviation angle.

16. The method of claim 11, further comprising defining a virtual walking boundary and providing an alert in response to detecting deviation beyond the virtual walking boundary.

17. A non-transitory computer-readable medium storing instructions that, when executed by one or more processors of a virtual reality walking balance assessment system, cause the virtual reality walking balance assessment system to:
present, by a head-mounted display, an interactive virtual environment to a participant during over-ground walking under a walking test protocol that specifies a plurality of visual scene conditions and specifies a first walking surface condition and a second walking surface condition that is more compliant than the first walking surface condition;
obtain head-tracking data representing motion of the head-mounted display while the participant walks under the first walking surface condition and the second walking surface condition; and
compute, from the head-tracking data, one or more gait balance metrics and one or more sensory contribution ratios indicative of relative contributions of visual input, vestibular input, and somatosensory input to walking balance.

18. The non-transitory computer-readable medium of claim 17, wherein the instructions further cause the virtual reality walking balance assessment system to display the one or more gait balance metrics on a display.

19. The non-transitory computer-readable medium of claim 17, wherein the instructions further cause the virtual reality walking balance assessment system to store or export the one or more gait balance metrics to a secure cloud-based database.

20. The non-transitory computer-readable medium of claim 17, wherein the instructions further cause the virtual reality walking balance assessment system to generate a recommendation for a rehabilitation strategy based on the one or more sensory contribution ratios.

21. A virtual reality rehabilitation system for improving walking balance in a participant, comprising:
a head-mounted display configured to present an interactive virtual environment to the participant during over-ground walking and to generate head-tracking data representing motion of the participant during the over-ground walking;
a walking surface arrangement configured to provide a plurality of walking surface conditions having different compliance characteristics; and
one or more processors configured to obtain movement data from the participant during the over-ground walking, determine from the movement data one or more gait balance metrics and one or more sensory contribution ratios indicative of relative reliance on visual input, vestibular input, and somatosensory input during walking, identify a sensory deficit profile for the participant based on the one or more gait balance metrics and the one or more sensory contribution ratios, and select or modify one or more rehabilitation tasks presented within the interactive virtual environment based on the sensory deficit profile.

22. The system of claim 21, wherein the interactive virtual environment comprises a virtual hallway.

23. The system of claim 21, wherein the rehabilitation tasks comprise walking under altered visual scene conditions.

24. The system of claim 23, wherein the altered visual scene conditions comprise a stable scene, a dark scene, and a rotating scene.

25. The system of claim 21, wherein the rehabilitation tasks comprise walking on a foam surface.

26. The system of claim 21, wherein the rehabilitation tasks comprise simultaneous head movements during walking.

27. The system of claim 26, wherein the head movements comprise horizontal head turns, vertical head turns, diagonal head turns, or combinations thereof.

28. The system of claim 21, wherein the rehabilitation tasks include dual-task walking activities in which the participant performs a cognitive task simultaneously with walking.

29. The system of claim 21, wherein the one or more processors are configured to progressively modify the one or more rehabilitation tasks according to participant performance.

30. The system of claim 29, wherein progressively modifying the one or more rehabilitation tasks comprises increasing at least one of walking speed, walking duration, visual complexity, head movement frequency, or surface compliance.

31. The system of claim 21, wherein the one or more processors are configured to provide real-time feedback regarding walking performance during execution of the one or more rehabilitation tasks.

32. The system of claim 31, wherein the real-time feedback comprises visual feedback, auditory feedback, haptic feedback, or combinations thereof.

33. The system of claim 21, wherein the one or more processors are configured to determine changes in the one or more gait balance metrics over multiple rehabilitation sessions and to modify the one or more rehabilitation tasks over time based on the determined chan
